(19) United States
(12) Patent Application Publication
(10) Pub. No.: US 2010/0210695 A1
(43) Pub. Date: Aug. 19, 2010

(54) COMPOSITIONS AND METHODS FOR TREATING SKIN DISORDERS

(75) Inventor: John R. Didsbury, Raleigh, NC (US)

Correspondence Address: MYERS BIGEL SIBLEY & SAJOVEC, PO BOX 37428, RALEIGH, NC, 27627, US

(73) Assignee: DARA BIOSCIENCES, INC., Raleigh, NC (US)

(21) Appl. No.: 12/666,232

(22) PCT Filed: Jun. 19, 2008
(86) PCT No.: PCT/US2008/007640

§ 371 Date: Jan. 25, 2010

Related U.S. Application Data
(60) Provisional application No. 60/945,682, filed on Jun. 22, 2007.

(51) Int. Cl. A61K 31/44; A61K 31/225; A61P 17/00

(52) U.S. Cl. 514/355; 514/547

(57) ABSTRACT

The present invention is directed to pharmaceutical compositions formulated for topical administration comprising an inhibitor of carnitine palmitoyl transferase (CPT1) and methods for treating skin disorders, such as psoriasis, by administration of a CPT1 inhibitor. In particular embodiments, the invention provides a pharmaceutical composition formulated for topical administration comprising the CPT1 inhibitor ST1326. In other embodiments, the invention provides a method of treating a skin disorder, such as psoriasis by administration of the CPT1 inhibitor ST1326.

### RELATED APPLICATION INFORMATION

This application claims the benefit of priority from U.S. Provisional Patent Application Ser. No. 60/945,682, filed Jun. 22, 2007, which is incorporated herein by reference in its entirety.

### Field of the Invention

The present invention is directed to pharmaceutical compositions and methods for treating skin disorders. In particular, the invention is directed to pharmaceutical compositions comprising an inhibitor of carnitine palmitoyl transferase I (CPT1) and use thereof to treat skin disorders.

### BACKGROUND OF THE INVENTION

U.S. Pat. Nos. 6,444,701 B1 and 6,369,073 to Giannessi et al. and Giannessi et al. (J. Med. Chem. 44:2383-2386 (2001)) describe amino carnitine derivatives that are inhibitors of carnitine palmitoyl transferase I (CPT1) and use thereof for treatment of hyperglycemia, diabetes, and pathologies related thereto such as heart failure and ischemia.

Giannessi et al. (J. Med. Chem. 46:303-309 (2003)) also describes aminocarnitine derivatives that are CPT1 inhibitors and their use as antiketotic and antidiabetic agents.

PCT Publication WO 2004/026405 to Nieland et al. describes inhibitors of fatty acid oxidation for the prophylaxis and/or treatment of chronic and/or atopic skin diseases.

### SUMMARY OF THE INVENTION

The present invention is directed to pharmaceutical compositions formulated for topical administration comprising an inhibitor of carnitine palmitoyl transferase (CPT1) and methods for treating skin disorders, such as psoriasis, by administration of a CPT1 inhibitor.

In particular embodiments, the CPT1 inhibitor is a compound of Formula (I)

X⁺—CH₂—CH(Z)—CH₂—Y⁻ Formula (I)

wherein: X⁺is N⁺(R₁,R₂,R₃) or P⁺(R₁,R₂,R₃);

wherein (R₁,R₂,R₃), being the same or different, are selected from the group consisting of hydrogen, a C₁-C₉ straight or branched alkyl group, —CH═NH(NH₂), —NH₂, and —OH; or one or more of R₁, R₂ and R₃, together with the nitrogen atom to which they are linked, form a saturated or unsaturated, monocyclic or bicyclic heterocyclic system; with the proviso that at least one of the R₁, R₂ and R₃ is different from hydrogen;

Z is selected from —OR₄, —OCOOR₄, —OCONH₄, —OCSNHR₄, —OCSOR₄, —NHR₄, —NHCOR₄, —NHCSR₄, —NHCOOR₄, —NHCSOR₄, —NHCONHR₄, —NHCSNHR₄, —NHSOR₄, —NHSONHR₄, —NHSO₂R₄, —NHSO₂NHR₄, and —SR₄,

wherein —R₄ is a C₁-C₂₀ saturated or unsaturated, straight or branched alkyl group, optionally substituted with an A₁ group, wherein A₁ is selected from the group consisting of a halogen atom, or an aryl, heteroaryl, aryloxy or heteroaryloxy group, said aryl, heteroaryl, aryloxy or heteroaryloxy groups being optionally substituted with one or more C₁ -C₂₀ saturated orunsaturated, straight or branched alkyl or alkoxy group and/or halogen atom;

Y⁻is selected from the group consisting of —COO⁻, PO₃H⁻, —OPO₃H⁻, and tetrazolate-5-yl

their (R,S) racemic mixtures, their single R or S enantiomers, or a pharmaceutically acceptable salt or prodrug thereof.

As another aspect of the invention, the CPT1 inhibitor is a compound of Formula (II)

(CH₃)₃N⁺CH₂CH(ZR)CH₂COO⁻ Formula (II)

wherein:

Z=ureido, carbamate, sulfonamide, or sulfamide moieties; and

R=C₇ to C₁₄ linear alkyl chains,

their (R,S) racemic mixtures, their single R or S enantiomers, or a pharmaceutically acceptable salt or prodrug thereof.

As a further aspect of the invention, the CPT1 inhibitor is a compound of Formula (III)

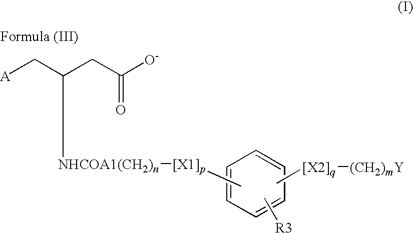

wherein:

A is selected between —N⁺(R R₁ R₂), —P⁺(R R₁ R₂), in which R, R₁, R₂ are the same or different and are selected from the group consisting of (C₁-C₂) alkyl, phenyl and phenyl-(C₁-C₂) alkyl; A1 is O or NH or is absent;

n is an integer number ranging from 0 to 20;

p is 0 or 1; q is 0, 1;

X1 is O or S;

X2 is O or S;

m is an integer number ranging from 1 to 20;

Y selected among H, phenyl and phenoxy; R3 is selected among H, halogen, linear or branched (C₁-C₄) alkyl and (C₁-C₄) alkoxy,

their (R,S) racemic mixtures, their single R or S enantiomers, or a pharmaceutically acceptable salts or prodrugs thereof.

In yet other embodiments, the CPT1 inhibitor is a compound of Formula (IV)

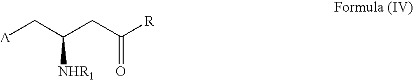

where:

A is selected among —N(R₂R₃), —N(R₂R₃R₄)⁺ and —C(R₂R₃R₄), in which the same or different R₂, R₃, R₄ are selected among H, alkyl C₁-C₂, phenyl, phenyl-alkyl C₁-C₂;

R is selected among —OH, —O^{θ}, linear or branched alkoxy C₁-C₄, optionally replaced by a carboxy or alkoxycarbonyl group C₁-C₄, or the group Y—Z, in which:

Y=—O—(CH₂)ₙ—O—, —O—(CH₂)ₙ—NH—, —S—(CH₂)ₙ—O—, —S—(CH₂)ₙ—NH—, where n is selected among 1, 2 and 3, or —O—(CH₂)ₙ—NH—, where n is selected among 0, 1, 2 and 3; and

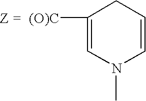

R₁ is selected among —COOR₅, —CONHR₅, —SOR₅, —SONHR₅, —SO₂R₅ and —SO₂NHR₅, in which

R₅ is a saturated or unsaturated, linear of branched alkyl C₁-C₂₀, replaced by aryl C₆-C₁₀, aryloxy heteroaryl C₆-C₁₀ heteroaryl C₄-C₁₀ containing 1 or more atoms selected among N, O and S, heteroaryloxy C₄-C₁₀ containing 1 or more atoms selected among N, O and S, in turn replaced by saturated or unsaturated, linear or branched alkyl or alkoxy C₁-C₂₀;

their (R,S) racemic mixtures, their single R or S enantiomers, their pharmaceutically acceptable salts and prodrugs thereof.

As still another aspect, the invention provides a method of treating a skin disorder in a mammalian subject comprising topically administering to the skin of the mammalian subject an effective amount of a CPT1 inhibitor, optionally R-4-trimethylammonium-3-[etradecylcarbamoyl)-aminobutyrate (ST1326).

As a further aspect, the invention provides a method of treating psoriasis in a mammalian subject comprising topically administering to the skin of the mammalian subject an effective amount of R-4-trimethylammonium-3-(tetradecylcarbamoyl)-aminobutyrate (ST1326) or a pharmaceutically acceptable salt or prodrug thereof in a pharmaceutically acceptable carrier.

In particular embodiments, the method further comprises administering a steroid to the mammalian subject.

The invention also provides for the use of a CPT1 inhibitor as described herein for the manufacture of a medicament to treat a skin disorder.

These and other aspects of the invention are set forth in more detail in the following description of the invention.

### DETAILED DESCRIPTION OF THE INVENTION

The present invention will now be described with reference to the accompanying drawings, in which preferred embodiments of the invention are shown. This invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Any feature of the invention that is specifically described herein can be included or omitted from the invention (e.g., can be disclaimed).

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

As used herein the term “consisting essentially of” (and grammatical variations) means that the composition, product or method does not comprise any elements that materially change the functioning of the composition, product of method other than those elements specifically recited. For example, a pharmaceutical composition of the invention “consisting essentially of” a CPT1 inhibitor, may also include a pharmaceutically acceptable excipient, a preservative, a diluent, a carrier, a dispersing agent, a moisturizer, a wetting agent, a thickening agent, and/or a penetration enhancer, and the like or any other substance that does not materially alter the pharmaceutical activity of the composition (e.g., inhibition of CPT1 and/or treatment of a skin disorder).

The pharmaceutical compositions of the present invention comprise, consist essentially of, or consist of an inhibitor of carnitine palmitoyl transferase 1 (CPT1 including CPT1L and/or CPT1M). It particular embodiments, the compound is an inhibitor of CPT1L and is optionally a selective inhibitor of CPT1L. In embodiments of the invention, the CPT1 inhibitor is a reversible CPT1 inhibitor.

The present invention can be used to treat any skin disorder, including without limitation, psoriasis, acne, actinic keratosis, atopic dermatitis, dermatomyositis, rosacea, urticaria, angioedema, seborrheic dermatitis, cutaneous atopy (e.g., eczema), Darrier's disease, xerosis, ichtyosis, pigmentation disorders, hyperkeratosis, mycosis fungoides, lichen planus, hyperplasia of the epidermis, and any combination thereof.

The present invention can be practiced with any CPT1 inhibitor, now known or later discovered, and a wide array of CPT1 inhibitors are known in the art.

U.S. Pat. Nos. 6,444,701 and 6,369,073 to Giannessi et al. and Giannessi et al. (J. Med. Chem. 44:2383-2386 (2001)) disclose a large number of aminocarnitine derivatives that are inhibitors of CPT1.

Accordingly, the compound can be an aminocarnitine derivative represented by the general formula:

X⁺—CH₂—CH(Z)—CH₂—Y⁻ Formula (I)

wherein: X⁺ is N⁺(R₁,R₂,R₃) or P⁺(R₁,R₂,R₃);

wherein (R₁,R₂,R₃), being the same or different, are selected from the group consisting of hydrogen, a C₁-C₉ straight or branched alkyl group, —CH—NH(NH₂), —NH₂, and —OH; or one or more of R₁, R₂ and R₃, together with the nitrogen atom to which they are linked, form a saturated or unsaturated, monocyclic or bicyclic heterocyclic system; with the proviso that at least one of the R₁, R₂ and R₃ is different from hydrogen;

Z is selected from —OR₄, —OCOOR₄, —OCONH₄, —OCSNHR₄, —OCSOR₄, —NHR₄, —NHCOR₄, —NHCSR₄, —NHCOOR₄, —NHCSOR₄, —NHCONHR₄, —NHCSNHR₄, —NHSOR₄, —NHSONHR₄, —NHSO₂R₄, —NHSO₂NHR₄, and —SR₄,

wherein —R₄ is a C₁-C₂₀ saturated or unsaturated, straight or branched alkyl group, optionally substituted with an A₁ group, wherein A₁ is selected from the group consisting of a halogen atom, or an aryl, heteroaryl, aryloxy or heteroaryloxy group, said aryl, heteroaryl, aryloxy or heteroaryloxy groups being optionally substituted with one or more C₁-C₂₀ saturated or unsaturated, straight or branched alkyl or alkoxy group and/or halogen atom;

Y⁻is selected from the group consisting of —COO⁻, PO₃H⁻, —OPO₃H⁻, and tetrazolate-5-yl;

their (R,S) racemic mixtures, their single R or S enantiomers, or their pharmaceutically acceptable salts or prodrugs.

In particular embodiments, the compound of Formula (I) is subject to the proviso that when Z is —NHCOR₄, X⁺is trimethylammonium, and Y is —COO−, then R₄ is C₂₀ alkyl.

In particular embodiments, the compound of Formula (I) is subject to the proviso that when Z is —NHSO₂R₄, X⁺is trimethylammonium, and Y⁻is —COO⁻, then R₄ is not tolyl.

In particular embodiments, the compound of Formula (I) is subject to the proviso that when Z is —NHR₄, X⁺is trimethylammonium and Y⁻is —COO⁻, then R₄ is not C₁-C₆ alkyl.

As examples of C₁-C₂₀ linear or branched alkyl group, methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl and eicosyl and their possible isomers are meant, such as for example isopropyl, isobutyl, tert-butyl.

Examples of C₁-C₂₀ linear or branched alkenyl group are methylene, ethylidene, vinyl, allyl, propargyl, butylene, pentylene, wherein the carbon-carbon double bond, optionally in the presence of other carbon-carbon unsaturations, can be situated in the different possible positions of the alkyl chain, which can also be branched within the allowed isomery.

Examples of (C₆-C₁₄) aryl group are phenyl, 1- or 2-naphthyl, anthryl, optionally substituted as shown in the general definitions above-mentioned.

Examples of heterocyclic groups thienyl, quinolyl, pyridyl, N-methylpiperidinyl, 5-tetrazolyl, optionally substituted as shown in the general definitions above-mentioned.

Halogen atoms include fluorine, chlorine, bromine, iodine.

The compounds of Formula (I) can also be in the form of inner salts.

In particular embodiments, the compounds comprise the compounds of Formula (I) wherein N⁺ (R₁,R₂,R₃) is trimethyl ammonium.

In other embodiments, the compounds comprise the compounds of Formula (I) wherein two or more of R₁, R₂ and R₃, together with the nitrogen atom to which they are linked, form a saturated or unsaturated, monocyclic or bicyclic heterocyclic system; for example morpholinium, pyridinium, pyrrolidinium, quinolinium, quinuclidinium.

In further representative embodiments, the compounds comprise the compounds of Formula (I) wherein R₁ and R₂ are hydrogen and R₃ is selected from the group consisting of —CH═NH(NH₂), —NH₂ and —OH.

Within particular embodiments of the present invention, the R₄ group can be a C₇-C₂₀ saturated or unsaturated, straight or branched alkyl group. In fact, it has been observed that a longer alkyl chain R₄ (>C₁₀) can significantly increase the selectivity against CPT1. Examples of R₄ groups include heptyl, octyl, nonyl, decyl, undecyl, dodecyl, tridecyl, tetradecyl, pentadecyl, hexadecyl, heptadecyl, octadecyl, nonadecyl and eicosyl.

Examples of Z groups are ureido (—NHCONHR₄), and carbamate (—NHCOOR₄, —OCONHR₄) groups.

In particular embodiments, compounds of Formula (I) comprise compounds wherein X⁺, R₁, R₂, R₃, have the above disclosed meanings, Z is ureido (—NHCONHR₄) or carbamate (—NHCOOR₄, —OCONHR₄), R₄ is a C₇-C₂₀, preferably a C₉-C₁₈ saturated or unsaturated, straight or branched alkyl group.

The compounds of Formula (I) have an asymmetry center on the carbon atom bound to a Z group. For the purposes of the present invention, each compound of Formula (I) can exist as a R,S racemic mixture or as separated R/S isomeric form.

The compounds of Formula (I) are quaternary ammonium or phosphonium derivatives containing a Y⁻ anionic group. Dependent on pH, each compound of Formula (I) can exist as an amphoion (inner salt) or as a compound wherein Y³¹ is present in the YH form. In such a case, X⁺ is salified with a pharmacologically acceptable acid. Formula (I) covers all these different possibilities.

Representative compounds of Formula (I) include but are not limited to:
R,S-4-trimethylammonium-3-(nonylcarbamoyl)-aminobutyrate;
R,S-4-quinuclidinium-3-(tetradecyloxycarbonyl)-oxybutyrate;
R,S-4-trimethylammonium-3-(nonylcarbamoyl)-oxybutyrate;
R,S-4-trimethylammonium-3-(nonyloxycarbonyl)-oxybutyric acid chloride;
R,S-4-trimethylphosphonium-3-(nonylcarbamoyl)-oxybutyrate;
R,S-4-trimethylammonium-3-(octyloxycarbonyl)-aminobutyrate;
R,S-4-trimethylammonium-3-(nonyloxycarbonyl)-aminobutyrate;
R,S-4-trimethylammonium-3-octyloxybutyrate;
R,S-4-trimethylammonium-3-tetradecyloxybutyrate;
R,S-1-guanidinium-2-tetradecyloxy-3-(tetrazolate-5-yl)-propane;
R,S-1-trimethylammonium-2-tetradecyloxy-3-(tetrazolate-5-yl)-propane;
R,S-3-quinuclidinium-2-(tetradecyloxycarbonyI)-oxy-1-propanephosphonate monobasic;
R,S-3-trimethylammonium-2-(nonylaminocarbonyl)-oxy-1-propanephosphonate monobasic;
R,S-3-pyridinium-2-(nonylaminocarbonyl)-oxy-1-propanephosphonic acid chloride;
R-4-trimethylammonium-3-(tetradecylcarbamoyl)-aminobutyrate;
R-4-trimethylammonium-3-(undecylcarbamoyl)-aminobutyrate;
R-4-trimethylammonium-3-(heptylcarbamoyl)-aminobutyrate;
R,S-4-trimethylammonium-3-(nonylthiocarbamoyl)-aminobutyrate;
R-4-trimethylammonium-3-(nonylcarbamoyl)-aminobutyrate;
S-4-trimethylammonium-3-(nonylcarbamoyl)-aminobutyrate;
S-4-trimethylammonium-3-(tetradecylcarbamoyl)-aminobutyrate;
R,S-4-trimethylammonium-3-tetradecylaminobutyrate;
R,S-4-trimethylammonium-3-octylaminobutyrate;
R,S-4-trimethylammonium-3-(decansulfonyl)aminobutyrate;
R,S-4-trimethylammonium-3-(nonylsulfamoyl)aminobutyrate;
S-4-trimethylammonium-3-(dodecansulfonyl)aminobutyrate;
R-4-trimethylammonium-3-(dodecansulfonyl)aminobutyrate;
S-4-trimethylammonium-3-(undecylsulfamoyl)aminobutyrate;
R-4-trimethylammonium-3-(undecylsulfamoyl)aminobutyrate;
R-4-trimethylammonium-3-(dodecylcarbamoyl)aminobutyrate;
R-4-trimethylammonium-3-(10-phenoxydecylcarbamoyl)aminobutyrate; R-4-trimethylammonium-3-(trans-.beta.-styrenesulfonyl) aminobutyrate.

In representative embodiments of the invention, the compound is R-4-trimethylammonium-3-(tetradecylcarbamoyl)-aminobutyrate (ST1326), R-4-trimethylammonium-3-(undecylcarbamoyl)-aminobutyrate (ST1327), R-4-trimethylammonium-3-(heptylcarbamoyl)-aminobutyrate (ST1328), S-4-trimethylammonium-3-(tetradecylcarbamoyl)-aminobutyrate (ST1340) and/or R-4-trimethylammonium-3-(dodecylcarbamoyl)aminobutyrate (ST1375).

The compounds of Formula (I) can be prepared by synthetic reactions that are well known in the art (see, e.g., U.S. Pat. Nos. 6,444,701 and 6,369,073 to Giannessi et al).

The compound can alternatively be an aminocarnitine derivative as described by Giannessi et al. (J. Med. Chem. 46:303-309 (2003)) represented by the general formula:

(CH₃)₃N⁺CH₂CH(ZR)CH₂COO⁻ Formula (II)

wherein:

Z=ureido, carbamate, sulfonamide, or sulfamide moieties; and

R=C₇ to C₁₄ linear alkyl chains.

The compounds of Formula (II) include both R and S forms. In particular embodiments, the compound is the (R) form of the ureido derivative (ZR═NHCONHR, R═C₁₄), the sulfonamidic derivative (ZR═NHSO₂R, R═C₁₂), or the sulfamidic derivative (ZR═NHSO₂NHR, R═C₁₁).

The CPT1 inhibitory compounds encompass pharmaceutically acceptable salts of the compounds described herein, including the compounds of Formula (I) and (II).

The term “pharmaceutically acceptable salts” refers to salts that retain the desired biological activity of the parent compound and do not impart undesired toxicological effects thereto.

Pharmaceutically acceptable base addition salts can be formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., (1977) “Pharmaceutical Salts,” J. of Pharma Sci. 66:1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from the respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids including, for example, with inorganic acids, such as hydrochloric acid, hydrobromic acid, sulfuric acid or phosphoric acid; with organic acids such as carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as naturally-occurring alpha-amino acids, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

Other CPT1 inhibitors include derivatives of 4-trimethylammonium 3-aminobutyrate and 4-trimethylphosphonium 3-aminobutyrate, for example, as described in PCT Publication WO 2008/015081 to Giannessi et al.

Accordingly, the compound can be a compound represented by the general formula:

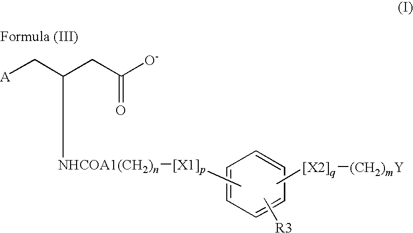

wherein:

A is selected between —N⁺ (R R₁ R₂), —P⁺ (R R₁ R₂), in which R, R₁, R₂ are the same or different and are selected from the group consisting of (C₁-C₂) alkyl, phenyl and phenyl-(C₁-C₂) alkyl; A1 is O or NH or is absent;

n is an integer number ranging from 0 to 20;

is 0 or 1 ; q is 0, 1;

X1 is O or S;

X2 is O or S;

m is an integer number ranging from 1 to 20;

Y selected among H, phenyl and phenoxy;

R3 is selected among H, halogen, linear or branched (C₁-C₄) alkyl and (C₁-C₄) alkoxy.

In embodiments of the invention, R, R₁ and R₂ are all methyl. In particular embodiments, m is an integer number ranging from 1 to 10, optionally from 4 to 8.

For the purposes of the present invention it is clarified that each of the products of Formula (III) can exist both as a racemic mixture R/S, and in the separate isomeric forms R and S.

The invention also encompasses pharmaceutically acceptable salts. Exemplary pharmaceutically acceptable salts of the compounds of Formula (III) are acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

Within the framework of the present invention, examples of the linear or branched (C₁-C₄) alkyl group, are understood to include methyl, ethyl, propyl and butyl and their possible isomers, such as, for example, isopropyl, isobutyl, and terbutyl.

The following are some representative compounds of Formula (III):
(R)-4-trimethylammonium-3-[[4-[(3-hexyloxy)-phenoxy]butyl]carbamoyl]-amino-butyrate (ST2425);
(R)-4-trimethylphosphonium-3-[[4-[(3-hexyloxy)-phenoxy]butyl]carbamoyl]-amino-butyrate (ST2452);
(R)-4-trimethylammonium-3-[[4-(heptyloxy)-phenyl]-carbamoyl]-amino-butyrate (ST2773);
(R)-4-trimethylammonium-3[[2-(benzyloxy)-benzyl]carbamoyl]-amino-butyrate (ST2790);
(R)-4-trimethylammonium-3-[[(4-benzyloxy-3-methoxy)-benzyl]carbamoyl]-amino-butyrate (ST2816);
(R)-4-trimethylammonium-3-[[4-[(2-hexyloxy)-phenoxy]butyl] carbamoyl]-amino-butyrate (ST4005);
(R)-4-trimethylammonium-3-[[4-[(3-hexyloxy)-phenoxy]propil] carbamoyl]-amino-butyrate (ST4024); and
(R)-4-trimethylammonio-3-[[3-(hexyloxy)phenoxy]acetyl]-amino-butyrate (ST4004).

The CPT1 inhibitor can further be an aminobutanoic acid derivative, for example, as described by international publication WO 2006/092204 to Giannessi et al.

Accordingly, the compound can be a compound represented by the general formula:

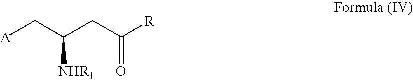

where:

A is selected among —N(R₂R₃), —N(R₂R₃R₄)^{*} and —C(R₂R₃R₄), in which the same or different R₂, R₃, R₄ are selected among H, alkyl C₁- C₂, phenyl, phenyl-alkyl C₁-C₂;

R is selected among —OH, —O^{θ}, linear or branched alkoxy C₁-C₄, optionally replaced by a carboxy or alkoxycarbonyl group C₁-C₄, or the group Y—Z, in which:

Y═—O—(CH₂)ₙ—NH—, —S—(CH₂)ₙ—O—, —S—(CH₂)ₙ—NH—, where n is selected among 1, 2 and 3, or —O—(CH₂)ₙ—NH—, where n is selected among 0, 1, 2 and 3; and

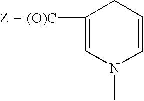

R₁ is selected among —COOR₅, —CONHR₅, —SOR₅, —SONHR₅, —SO₂R₅ and —SO₂NHR₅, in which

R₅ is a saturated or unsaturated, linear of branched alkyl C₁-C₂₀, replaced by aryl C₆-C₁₀, aryloxy C₆-C₁₀, heteroaryl C₄-C₁₀ containing 1 or more atoms selected among N, O and S, heteroaryloxy C₄-C₁₀ containing 1 or more atoms selected among N, O and S, in turn replaced by saturated or unsaturated, linear or branched alkyl or alkoxy C₁-C₂₀.

In particular embodiments, when A is —N(R₂R₃R₄)⁺ and R₂, R₃ and R₄ are the same and are alkyls, R is different from —OH or —O^{θ}.

In embodiments of the invention, R₁ is —CONHR₅ and R₅ is a linear or branched alkyl, saturated or unsaturated, containing between 7 and 20 carbon atoms. Exemplary R₅ groups are therefore selected among heptyl, octyl, nonyl, decyl, undecyl, dodecyl, tridecyl, tetradecyl, pentadecyl, hexadecyl, heptadecyl, octadecyl, nonadecyl and eicosyl.

In embodiments of the invention, R₂ or R₃ or both are methyl.

Depending on the meanings of the radicals A, R₁, R₂, R₃, R₄, R₅, Y and Z, in the compounds of Formula (IV), one or more chiral centers (on carbon or nitrogen atoms) may be present. For the purposes of the present invention it is pointed out that each of the products of Formula (IV) can exist both as a racemic mixture R/S, and in the separate isomeric forms R and S.

The compounds of Formula (IV), in which A is —N(R₂R₃R₄)⁺ and R is different from —OH and —O^{θ}, can exist as salts with pharmaceutically acceptable anions. These anions are here identified by the radical X⁻.

The compounds of Formula (IV) in which A is —N(R₂R₃) can exist as internal salts, as salts with pharmaceutically acceptable acids and also in anionic form without a positive net charge on the nitrogen in group A.

The compounds of Formula (IV) in which A does not contain nitrogen can exist in neutral or anionic form.

The present invention covers all these different possibilities of salification for the compounds of Formula (IV).

Representative pharmaceutically acceptable salts (I) are acid addition salts formed with pharmaceutically acceptable acids like hydrochloride, hydrobromide, hydroiodide, sulfate or bisulfate, phosphate or hydrogen phosphate, acetate, benzoate, succinate, fumarate, maleate, lactate, citrate, tartrate, gluconate, methanesulfonate, benzenesulfonate, and para- toluenesulfonate salts.

Suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Sodium salts are particularly preferred.

The following are representative compounds of Formula (IV): (R)-4-(dimethyl amino)-3-(tetradecyl carbamoyl)-methyl aminobutyrate; (R)-4-(dimethyl amino)-3-(tetradecyl carbamoyl)-aminobutyric acid; (R)-4-(trimethyl amino)-3-(tetradecyl carbamoyl)-methyl aminobutyrate chloride; (R)-4-trimethylammonium-3-(tetradecylcarbamoyl)-amino-butyrate of {2[-(N-methyl-(1,4-dihydro-pyridine)-3-yl)carbonyl]-amino}ethyl iodide; and (R)-4-trimethylammonium-3 (tetradecylcarbamoyl)-amino-butyrate of -3-(methoxycarbonyl)-propyl bromide.

The compounds of Formula (IV) can be prepared using reactions known in the state of the art and in 2006/092204. Examples of these reactions are reported in International publication WO 99/59957, Eur. J. Org. Chem. 2003, 4501-4505, Eur. J. Med. Chem. 39 (2004), 715-727 and Helv. Chim. Acta 1996, 79, 1203-1216.

Compounds of the formulae herein include those having quaternization of any basic nitrogen-containing group therein.

The discussion herein is, for simplicity, provided without reference to stereoisomerism. Those skilled in the art will appreciate that the compounds of formulae described herein may contain one or more asymmetric centers and thus occur as racemates and racemic mixtures, single optical isomers, individual diastereomers, and diastereomeric mixtures. All such isomeric forms of these compounds are expressly included in the present invention.

Similarly, compounds of the invention containing a double bond can exist in the form of tautomers and geometric isomers, which can be readily separated and recovered by conventional procedures. Such isomeric forms are included in the scope of this invention.

Other CPT1 inhibitors include without limitation other aminocarnitine derivatives, long chain alkyloxy- and aryloxy-substituted phosphinyloxy derivatives of carnitine, including long chain alkoxy- and aryloxy-substituted 3-carboxy-2-phosphinyloxy-1-propanaminium hydroxide inner salt derivatives (for example, SDZ-CPI-975), see, e.g., EP 0 574 355 B1 to Anderson et al.; and Deems et al., (1998) Am J. Physiol. 274 (Regulatory Integrative Comp. Physiol. 43): R524-528. Other CPT1 inhibitors are oxirane derivatives. Examples of oxirane derivatives include oxirane carboxylates such as methyl palmoxirate (Rupp et al., (2002) Herz 27:621-636), etomoxir and etomoxir derivatives, clomoxir, 2-(5-(4-chlorophenyl)pentyl)oxirane-2-carboxylate (POCA), and 2-tetradecylglycidate (TDGA) (see, e.g., Wolf, “Possible New Therapeutic Approach in Diabetes Mellitus by Inhibition of Carnitine Palmitoyltransferase 1 (CPT1), Pathogenesis and Management of Human Diabetes Mellitus, Workshop at the 23^{rd} Annual Meeting of the European Society for Clinical Investigation 1989, Athens, Greece; Hormone and Metabolic Research Supplement Series Volume No. 26); Ratheiser et al., (1991) Metabolism 40:1185-1190; and Anderson et al., (1995) J. Med. Chem 38:3448-3450; Anderson, (1998) Current Pharmaceutical Design 4:1-15); and U.S. Patent Publication 2005/0004173 to Henkel et al. (arylalkyl- and aryloxyalkyl-substituted oxirane carboxylic acids). Oxirane carboxylic acids are also described in U.S. Pat. No. 6,479,676 to Wolf; U.S. Pat. No. 4,946,866 to Wolf; U.S. Pat. No. 4,430,339 to Eistetter et al.; U.S. Pat. No. 4,324,796 to Eistetter et al.; U.S. Pat. No. 4,788,306 to Schiehser et al.; U.S. Pat. No. 4,334,089 to Kraas et al.; U.S. Pat. No. 6,013,666 to Jew et al.; U.S. Pat. No. 5,739,159 to Wolf, and U.S. Pat. No. 4,788,304 to Marshall et al.

Further CPT1 inhibitors include but are not limited to 4-THA (2-hydroxy-3-propyl-4-[6-(tetrazol-5-yl)hexyloxy]acetophenone; Biochem. J. (1988) 252:409-414); 2-hydroxypropionic acid derivatives (U.S. Pat. No. 6,030,993 to Jew et al.), aminocarnitines and acylaminocarnitines (e.g., decanoyl-DL-amiocarnitine and palmitoyl-DL-aminocarnitine) as described by Jenkins et al., (1986) Proc. Natl. Acad. Sci 83:290-294), emeriamine (see, e.g., Kanamaru et al., Emeriamine: A new inhibitor of long chain fatty acid oxidation and its antidiabetic activity, Novel Microbial Products for Medicine and Agriculture, editors A. L. Demain et al., 135-144 (1989)), and acylamidomorpholinium carnitine analogues (see, e.g., Savle et al.(1999) Bioorganic & Medicinal Chemistry Letters 9:3099-3102).

Additional compounds that inhibit CPT1, including SDZ-269-456 and SDZ-267-597, are described by Anderson, (1998) Current Pharmaceutical Design 4:1-15. Further examples of CPT1 inhibitors include glibenclamide (Lehtihet et al., (2003) Am. J., Physiol. Endocrinol. Metabol. 185: E438-446), S-15176, metoprolol, perhexiline, trimetazidine, oxfenicine, and amiodarone (Rupp et al., (2002) Herz 27:621-636), a glycidic acid such as 2-tetradecyl-glycidate (TDGA), doxorubicin, ranolazine, and oxamic acid such as Ro25-087, an acyl-tetrahedra intermediate such as SDZ 265506 or hemipalmitoylcarnitinium or a (+)-acylcarnitine such as (+)-octenyl-carnitine or (+)-palmitoylcarnitine.

The compound can further be a pharmaceutically acceptable salt of any of the foregoing.

The pharmaceutical composition can comprise two or more different CPT1 inhibitors, and the methods of the invention can be practiced by administering two or more different CPT1 inhibitors, which can be formulated in the same pharmaceutical composition or separate compositions.

In embodiments of the invention, the composition does not comprise etomoxir. In embodiments of the invention, the composition does not comprise an arylalkyl or aryloxyalky-substituted oxirane carboxylic acid. In embodiments of the invention, the methods do not comprise administration of etomoxir. In embodiments of the invention, the methods do not comprise administration of an arylalkyl or aryloxyalky-substituted oxirane carboxylic acid.

In representative embodiments, the compound can be a prodrug that is converted to the active compound (e.g., as described above) in vivo. For example, the compound can be modified to enhance cellular permeability (e.g., by esterification of polar groups) and then converted by cellular enzymes to produce the active agent. Methods of masking charged or reactive moieties as a prodrug are known by those skilled in the art (see, e.g., P. Korgsgaard-Larsen and H. Bundgaard, A Textbook of Drug Design and Development, Reading U.K., Harwood Academic Publishers, 1991).

The term “prodrug” refers to compounds that are transformed in vivo to yield the parent compound of the above formulae, for example, by hydrolysis in blood, see, e.g., T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Exemplary prodrugs include a prodrug that is metabolized in vivo by a subject to an active drug having an activity of the compounds as described herein. For example, those skilled in the art will appreciate that a prodrug of a compound comprising an indole nitrogen can be an ester, such as a urethane ester. The term “pharmaceutically acceptable prodrug” (and like terms) as used herein refers to those prodrugs of the compounds of the present invention which are, within the scope of sound medical judgment, suitable for use in contact with the tissues of humans and/or other animals without undue toxicity, irritation, allergic response and the like, commensurate with a reasonable risk/benefit ratio, and effective for their intended use, as well as the zwitterionic forms, where possible, of the compounds of the invention.

In particular embodiments of the invention, the CPT1 inhibitor is an inhibitory nucleic acid such as an interfering RNA (RNAi) including short interfering RNAs (siRNA), an antisense nucleic acid, or a ribozyme directed against CPT1. The nucleic acid sequences of a number of CPT1 molecules are known, which facilitates the synthesis of inhibitory oligonucleotides to reduce the activity of these molecules, see, e.g., Genbank Accession No. NM_{—}001876 (CPT1L) and Genbank Accession No. NM_{—}004377 (CPT1M).

In particular embodiments, the CPT1 inhibitor is an antibody or antibody fragment that binds to CPT1 and reduces the activity thereof and/or interaction with binding partners. The antibody or antibody fragment is not limited to any particular form and can be a polyclonal, monoclonal, bispecific, humanized, chimerized antibody or antibody fragment and can further be a Fab fragment, single chain antibody, and the like.

Further, the CPT1 inhibitor can be a nucleic acid mimetic that inhibits CPT1 activity. A nucleic acid mimetic is an artificial compound that behaves similarly to a nucleic acid by having the ability to base-pair with a complementary nucleic acid. Non-limiting examples of mimetics include peptide nucleic acids and phosphorothionate mimetics. Another example of a mimetic is an aptamer, which binds to and inhibits the target molecule in a manner similar to an antibody or small molecule inhibitor.

The pharmaceutical compositions of the present invention can optionally be administered in conjunction with other therapeutic agents, for example, other therapeutic agents useful in the treatment of the skin disorder. For example, the compounds of the invention can be administered in conjunction with an inhibitor of malonyl CoA, an anti-inflammatory agent including steroids and/or non-steroidal compounds, a local anesthetic, another inhibitor of fatty acid oxidation (e.g. malonyl CoA decarboxylase inhibitors or other CPT1 inhibitors), a Vitamin D analogue (e.g. calcipotriene), lnfliximab, Adalimumab, Etanercept, Alefacept, Efalizumab, an immunosuppressant (e.g. tacrolimus), a phosphodiesterase-IV inhibitor (e.g. CC-10004), JB-991, AN-0128, AN-2728, a retinoid (e.g. tazarotene), anthralin, salicylic acid, an anti-IL12 antibody, an anti-IL23 antibody, an anti-IL15 antibody, coal tar, dithranol, urea, Mahonia aquifolium, a B vitamin or a derivative thereof (e.g., vitamin B12 or a derivative thereof), an antibiotic, an antimycotic, an immunomodulator (e.g., methotrexate, cyclosporine), and/or systemic treatment with fumaric acid, fumaric acid esters and/or blockers of arachidonic acid (e.g., omega-3 fatty acids).

The additional therapeutic agent(s) can be administered concurrently with the compound of the invention, in the same or different formulations. As used herein, the word “concurrently” means sufficiently close in time to produce a combined effect (that is, concurrently can be simultaneously, or it can be two or more events occurring within a short time period before or after each other). Further, the additional therapeutic agent can be administered by the same or different route as the CPT1 inhibitor.

Optionally, the pharmaceutical compositions of the invention are used in rotation with other treatment regimes to avoid the desensitization effect that often occurs with psoriasis and other skin diseases.

Exemplary malonyl CoA decarboxylase inhibitors include without limitation those described in U.S. Patent Publications 2004/0082576, 2004/0092503, and 2004/0087627 (Arrhenius et al.), the cyanoamide compounds described in U.S. Patent Publication 2005/0026945 (Kafka et al.), the piperidine compounds described in 2005/0032828 (Cheng et al.), the heterocyclic compounds described in U.S. Patent Publication 2005/0026969 (Cheng et al.), the cyanoguanidine-based azole compounds described in U.S. Patent Publication 2005/0032824 (Cheng et al.), CBM-300864, CBM-302280, CBM-302106, CBM-301940, CBM-302276, CBM-302342, CBM-302386, CBM-302075, CBM-302167, CBM-302189, CBM-302244, CBM-302052, and any combination thereof.

Additional fatty acid oxidation inhibitors include without limitation inhibitors of a fatty acid binding protein (e.g., psoriasis associated FABP), phospholipase A, lipoprotein lipase, hormone sensitive lipase, monoacylglycerol-lipase, acyl-CoA synthetase, carnitine-acylcarnitine-translocase, CPT2, acyl-CoA-dehydrogenase, enoyl-CoA-hydratase, L-3-hydroxyacyl-CoA-dehydrogenase, /or 3-ketoacyl-CoA thiolase, and any combination thereof.

Suitable steroids (e.g., corticosteroids) include without limitation betamethasone dipropionate, clobetasol propionate, diflorasone diacetate, halobetasol propionate, amcinonide, dosoximetasone, fluocinonide, halcinonide, mometasone furoate, betamethasone valerate, fluticasone propionate, triamcinolone acetonide, fluocinolone acetonide, flurandrenolide, memetasone furoate, desonide, fluticasone propionate, hydrocortisone butyrate, hydrocortisone valerate, alclometasone dipropionate, flumethasone pivalate, hydrocortisone, hydrocortisone acetate, and any combination thereof.

The invention also encompasses methods of treating skin disorders in a subject by administering (e.g., topically administering to the skin) a CPT1 inhibitor or a composition or pharmaceutical composition comprising, consisting essentially of, or consisting of the same. The mode of administration can be any suitable route that achieves the desired affects without undue side effects, such as toxicity, including but not limited to, topical, intravenous, intra-arterial, intraperitoneal, oral, buccal (e.g., sub-lingual), subcutaneous, transdermal, inhalation (via the mouth and/or nose), intramuscular, intra-vaginal, rectal administration and the like.

The invention also provides for the use of an active compound as described herein for the manufacture of a medicament to treat a skin disorder.

In representative embodiments, the skin disorder is psoriasis. In other representative embodiments, the pharmaceutical composition comprises R-4-trimethylammonium-3-(tetradecylcarbamoyl)-aminobutyrate (ST1326). In still other representative embodiments, the invention is practiced to topically administer to the skin of a mammalian subject an effective amount of a pharmaceutical composition comprising R-4-trimethylammonium-3-(tetradecylcarbamoyl)-aminobutyrate (ST1326) in pharmaceutically acceptable carrier (e.g., to treat psoriasis). Optionally, the subject is treated concurrently or in rotation with a corticosteroid.

When referring to a skin disorder, the word “skin” is meant to include any layer(s) of the skin in which a skin disorder may occur, extend to and/or reside, including that on limbs, trunk, head, as well as mucosa, etc. Thus the word “skin” is intended to include, but not be limited to, the epidermal and/or dermal layers, and may also include the underlying subcutaneous tissue.

The present invention finds use in research as well as veterinary and medical applications. Suitable subjects include both avians and mammals. The term “avian” as used herein includes, but is not limited to, chickens, ducks, geese, quail, turkeys and pheasants. The term “mammal” as used herein includes, but is not limited to, humans, non-human primates, cattle, sheep, goats, pigs, horses, cats, dog, rabbits, rodents (e.g., rats and/or mice), etc. In particular embodiments, the subject is a human subject that has been diagnosed with or is considered at risk for a skin disorder such as psoriasis. Human subjects include neonates, infants, juveniles, and/or adults. In other embodiments, the subject used in the methods of the invention is an animal model of a skin disorder such as psoriasis.

In particular embodiments of the invention, the subject is a subject “in need of” the methods of the present invention, e.g., in need of the therapeutic effects of the inventive methods. For example, the subject can be a subject that has been diagnosed with or is considered at risk for a skin disorder (e.g., psoriasis), and the methods of the invention are practiced on the subject as a method of prophylactic or therapeutic treatment.

As used herein, an “effective amount” refers to an amount of a compound or pharmaceutical composition that is sufficient to produce a desired effect, which is optionally a therapeutic effect (i.e., by administration of a therapeutically effective amount). For example, an “effective amount” can be an amount that is sufficient to treat a skin disorder such as psoriasis.

A “therapeutically effective” amount as used herein is an amount that provides some improvement or benefit to the subject. Alternatively stated, a “therapeutically effective” amount is an amount that provides some alleviation, mitigation, delay and/or decrease in at least one clinical symptom and/or prevent the onset or progression of at least one clinical symptom (e.g., reduction of inflammation present in psoriasis). Clinical symptoms associated with the disorders that can be treated by the methods of the invention are well-known to those skilled in the art. Further, those skilled in the art will appreciate that the therapeutic effects need not be complete or curative, as long as some benefit is provided to the subject.

By the terms “treat,” “treating” or “treatment of” (or grammatically equivalent terms) it is meant that the severity of the subject's condition is reduced or at least partially improved or ameliorated and/or that some alleviation, mitigation or decrease in at least one clinical symptom is achieved (e.g., in the severity and/or extent of skin lesions, patient discomfort), and/or there is a delay in the progression of the condition and/or prevention or delay of the onset of a disease or illness. Thus, the terms “treat,” “treating” or “treatment of” (or grammatically equivalent terms) refer to both prophylactic and therapeutic treatment regimes.

The invention encompasses pharmaceutical compositions formulated for topical administration comprising one or more compounds as described herein in a pharmaceutically acceptable carrier.

By “pharmaceutically acceptable” it is meant a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

The formulations of the invention can optionally comprise other medicinal agents, pharmaceutical agents, excipients, carriers, dispersing agents, diluents, humectants, moisturizers, wetting agents, thickening agents, penetration enhancers, preservatives, and the like.

In particular embodiments, the excipient comprises petroleum jelly, was, oleyl alcohol, propylene glycol monostearate, propylene glycol monopalmitostearate, isopropyl laureate, isopropyl myristate, isopropyl palmitate, isopropyl stearate, ethyl myristate, propyl myristate, butyl myristate, ethyl oleate, cetylstearyl alcohol, lanolin alcohol, paraffin oil, or any combination thereof.

The compositions of the invention can be formulated for topical administration in a pharmaceutical carrier in accordance with known techniques. See, e.g., Remington, The Science And Practice of Pharmacy (20^{th} edition, 2000). Suitable nontoxic pharmaceutically acceptable topical carriers will be apparent to those skilled in the art of topical pharmaceutical formulations (see, e.g., Remington's Pharmaceutical Sciences (Maack Publishing Co., Easton latest edition). Further, it will be understood by those skilled in the art that the choice of suitable carriers, absorption enhancers, humectants, adhesives, etc., will typically depend on the nature of the active compound and the particular topical formulation.

Topical formulations are known in the art. Suitable pharmaceutical compositions for topical administration include but are not limited to a lotion, liquid, cream, ointment, salve, emulsion, milk, powder, impregnated pad, solution, spray, suspension or gel. Further, the pharmaceutical composition can take the form of a shampoo, conditioner, hair tonic, hair spray, or hair foam. The active compound may be present as a suspension or a solution.

The active compound can optionally be formulated for extended and/or controlled release as is known in the art, e.g., as lipid or polymeric microspheres or nanospheres or vesicles, or a polymeric patch or hydrogel.

To extend shelf life, preservatives can optionally be added to the pharmaceutical composition. Suitable preservatives include but are not limited to benzyl alcohol, parabens, thimerosal, chlorobutanol and benzalkonium chloride, and combinations of the foregoing. The concentration of the preservative will vary depending upon the preservative used, the compound being formulated, the formulation, and the like. In representative embodiments, the preservative is present in an amount of 2% by weight or less.

In particular embodiments, the pharmaceutical composition is administered to the subject in an effective amount, optionally, a therapeutically effective amount (each as described hereinabove). Dosages of pharmaceutically active compositions can be determined by methods known in the art, see, e.g., Remington's Pharmaceutical Sciences (Maack Publishing Co., Easton, Pa; 18^{th} edition, 1990).

A therapeutically effective amount will vary with the age and general condition of the subject, the severity of the condition being treated, the particular compound or composition being administered, the duration of the treatment, the nature of any concurrent treatment, the carrier used, and like factors within the knowledge and expertise of those skilled in the art. As appropriate, a therapeutically effective amount in any individual case can be determined by one of ordinary skill in the art by reference to the pertinent texts and literature and/or by using routine experimentation (see, e.g., Remington, The Science and Practice of Pharmacy (20^{th} ed. 2000)).

The concentration of the active compound in the topical formulation is generally high enough to permit delivery of a therapeutically effective amount, but not so high as to cause unwanted side effects. In particular embodiments, the concentration is between about 0.05% and about 20%. In other embodiments, the concentration is between about 1% and about 6%.

In particular embodiments, the concentration is between about 0.1% and 20%, optionally administered at least once a day and during the period of time sufficient to achieve a therapeutic effect. In other variations, the concentration is between about 0.1% and about 15%; or between about 0.1% and about 12%; or between about 0.1% and about 20%; or between about 0.1% and about 15%; or between about 0.1% and about 12%; or between about 0.1% and about 10%; or between about 0.1% and about 8%; or between about 0.1% and about 4%; or between about 0.1% and about 2%; or between about 0.1% and about 1%; or between about 0.5% and about 10%; or between about 1% and about 10%; or between about 2% and about 20%; or between about 2% and about 15%; or between about 2% and about 12%; or between about 2% and about 10%; or between about 4% to about 20%; or between about 4% to about 15%; or between about 4% to about 12%; or between about 4% to about 10%; or between about 6% to about 10%; or between about 8% to about 10%; or between about 0.1 and about 5%; or between about 0.1% and about 4%; or between about 0.5% and about 5%; or between about 1% and about 4%; or between about 2% and about 4%; or between about 1% and about 3%; or between about 1.5% and about 3%; or between about 2% and about 3%; or between about 0.05% and about 10%; or between about 0.05% and about 8%; or between about 0.05% and about 4%; or between about 0.05% and about 4%; or between about 0.05% and about 3%.

The composition can be administered for a sustained period, such as at least about one month, at least about 2 months, at least about 3 months, at least about 6 months, or at least about 12 months or longer.

Other dosing schedules may also be followed. For example, the frequency of the administration may vary. The dosing frequency can be a once weekly dosing. The dosing frequency can be a once daily dosing. The dosing frequency can be more than once weekly dosing. The dosing frequency can be more than once daily dosing, such as any one of 2, 3, 4, 5, or more than 5 daily doses. The dosing frequency can be 3 times a day. The dosing frequency can be three times a week dosing. The dosing frequency can be a four times a week dosing. The dosing frequency can be a two times a week dosing. The dosing frequency can be more than once weekly dosing but less than daily dosing. The dosing frequency can be a once monthly dosing. The dosing frequency can be a twice weekly dosing. The dosing frequency can be more than once monthly dosing but less than one weekly dosing. The dosing frequency can intermittent (e.g., one daily dosing for 7 days followed by no doses for 7 days, repeated for any 14 day time period, such as 2 months, 4 months, 6 months or more). The dosing frequency can be continuous (e.g., one weekly dosing for continuous weeks).

In other embodiments, the methods of the invention can be carried out on an as-needed basis (e.g., by self-medication).

Any of the dosing frequencies can be used with any concentration of the active ingredient. Further, any of the dosing frequencies can employ any of the compounds described herein together with any of the dosages described herein, for treatment of any of the skin disorders described herein.

The foregoing is illustrative of the present invention, and is not to be construed as limiting thereof. The invention is defined by the following claims, with equivalents of the claims to be included therein.

1. A pharmaceutical composition formulated for topical administration comprising a compound that is an inhibitor of carnitine palmitoyl transferase I (CPT1) in a pharmaceutically acceptable carrier, wherein the composition does not comprise etomoxir.

2. The pharmaceutical composition of claim 1, comprising a compound of Formula (I)
X⁺—CH₂—CH(Z)—CH₂—Y⁻ Formula (I)
wherein: X⁺is N⁺(R₁,R₂,R₃) or P⁺(R₁,R₂,R₃);
wherein (R₁,R₂,R₃), being the same or different, are selected from the group consisting of hydrogen, a C₁-C₉ straight or branched alkyl group, —CH═NH(NH₂), —NH₂, and —OH; or one or more of R₁, R₂ and R₃, together with the nitrogen atom to which they are linked, form a saturated or unsaturated, monocyclic or bicyclic heterocyclic system; with the proviso that at least one of the R₁, R₂ and R₃ is different from hydrogen;
Z is selected from —OR₄, —OCOOR₄, —OCONH₄, —OCSNHR₄, —OCSOR₄, —NHR₄, —NHCOR₄, —NHCSR₄, —NHCOOR₄, —NHCSOR₄, —NHCONHR₄, —NHCSNHR₄, —NHSOR₄, —NHSONHR₄, —NHSO₂R₄, —NHSO₂NHR₄, and —SR₄,
wherein —R₄ is a C₁ -C₂₀ saturated or unsaturated, straight or branched alkyl group, optionally substituted with an A₁ group, wherein A₁ is selected from the group consisting of a halogen atom, or an aryl, heteroaryl, aryloxy or heteroaryloxy group, said aryl, heteroaryl, aryloxy or heteroaryloxy groups being optionally substituted with one or more C₁-C₂₀ saturated or unsaturated, straight or branched alkyl or alkoxy group and/or halogen atom;
Y⁻ is selected from the group consisting of —COO⁻, PO₃H⁻, —OPO₃H⁻, and tetrazolate-5-yl
their (R,S) racemic mixtures, their single R or S enantiomers, their pharmaceutically acceptable salts and prodrugs thereof.

3. The pharmaceutical composition of claim 2, with the proviso that when Z is —NHCOR₄, X⁺ is trimethylammonium and Y is —COO⁻, then R₄ is C₂₀ alkyl.

4. The pharmaceutical composition of claim 2, with the proviso that when Z is —NHSO₂R₄, X⁺ is trimethylammonium and Y⁻ is —COO⁻, then R₄ is not tolyl.

5. The pharmaceutical composition of claim 2, with the proviso that when Z is —NHR₄, X⁺ is trimethylammonium and Y⁻ is —COO⁻, then R₄ is not C₁-C₆ alkyl.

6. The pharmaceutical composition of claim 2, wherein R₁, R₂ and R₃ are methyl.

7. The pharmaceutical composition of claim 2, wherein the heterocyclic system formed by R₁, R₂ and R₃ together with nitrogen is morpholinium, quinuclidinium, pyridinium, quinolinium or pyrrolidinium.

8. The pharmaceutical composition of claim 2, wherein R₁, and R₂ are H, R₃ is —CH═NH(NH₂), —NH₂ or —OH.

9. The pharmaceutical composition of claim 2, wherein Z is ureido (—NHCONHR₄) or carbamate (—OCONHR₄), and R₄ is a C₇-C₂₀ saturated or unsaturated, straight or branched alkyl group.

10. The pharmaceutical composition of claim 9, wherein R₄ is a C₉- C₁₈ saturated or unsaturated, straight or branched alkyl group.

11. The pharmaceutical composition of claim 2, wherein the compound is R-4-trimethylammonium-3-[tetradecylcarbamoyl)-aminobutyrate (ST1326).

12. The pharmaceutical composition of claim 1, comprising a compound of Formula (II)
(CH₃)₃N³⁰CH₂CH(ZR)CH₂COO⁻ Formula (II)
wherein:
Z=ureido, carbamate, sulfonamide, or sulfamide moieties; and
R═C₇ to C₁₄ linear alkyl chains,
their (R,S) racemic mixtures, their single R or S enantiomers, their pharmaceutically acceptable salts and prodrugs thereof.

13. The pharmaceutical composition of claim 1, comprising a compound of Formula (III) 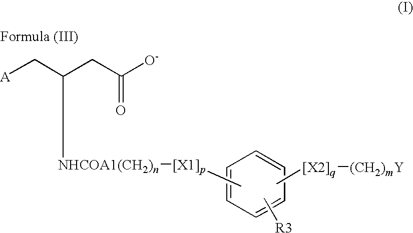
wherein:
A is selected between —N⁺ (R R₁ R₂), —P⁺ (R R₁ R₂), in which R, R₁, R₂ are the same or different and are selected from the group consisting of (C₁-C₂) alkyl, phenyl and phenyl-(C₁-C₂) alkyl; A1 is O or NH or is absent;
n is an integer number ranging from 0 to 20;
p is 0 or 1 : q is 0, 1;
X1 is O or S;
X2 is O or S;
m is an integer number ranging from 1 to 20;
Y selected among H, phenyl and phenoxy;
R3 is selected among H, halogen, linear or branched (C₁-C₄) alkyl and (C₁-C₄) alkoxy,
their (R,S) racemic mixtures, their single R or S enantiomers, their pharmaceutically acceptable salts and prodrugs thereof.

14. The pharmaceutical composition of claim 1, comprising a compound of Formula (IV) 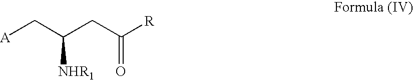
where:
A is selected among —N(R₂R₃), —N(R₂R₃R₄) and —C(R₂R₃R₄), in which the same or different R₂, R₃, R₄ are selected among H, alkyl C₁-C₂, phenyl, phenyl-alkyl C₁-C₂;
R is selected among —OH, —O^{θ}, linear or branched alkoxy C₁-C₄, optionally replaced by a carboxy or alkoxycarbonyl group C₁-C₄, or the group Y—Z, in which:
Y═—O—(CH₂)ₙ—O—, —O—(CH₂)ₙ—NH—, —S—(CH₂)ₙ—O—, —S—(CH₂)ₙ—NH—, where n is selected among 1, 2 and 3, or —O—(CH₂)ₙ—NH—, where n is selected among 0, 1, 2 and 3; and 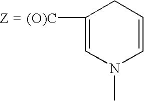
R₁ is selected among —COOR₅, —CONHR₅, —SOR₅, —SONHR₅, —SO₂R₅ and —SO₂NHR₅, in which
R₅ is a saturated or unsaturated, linear of branched alkyl C₁-C₂₀, replaced by aryl C₆-C₁₀, aryloxy heteroaryl C₄-C₁₀ containing 1 or more atoms selected among N, O and S, heteroaryloxy C₄-C₁₀ containing 1 or more atoms selected among N, O and S, in turn replaced by saturated or unsaturated, linear or branched alkyl or alkoxy C₁-C₂₀;
their (R,S) racemic mixtures, their single R or S enantiomers, their pharmaceutically acceptable salts and prodrugs thereof.

15. A method of treating a skin disorder in a mammalian subject comprising topically administering to the skin of the mammalian subject an effective amount of a pharmaceutical composition according to claim 1.

16. The method of claim 15, wherein the skin disorder is psoriasis, acne, actinic keratosis, atopic dermatitis, dermatomyositis, rosacea, urticaria, angioedema, seborrheic dermatitis, cutaneous atopy (e.g., eczema), Darrier's disease, xerosis,,ichtyosis, pigmentation disorders, hyperkeratosis, mycosis fungoides, lichen planus, hyperplasia of the epidermis, or any combination thereof.

17. The method of any of claim 15, wherein the compound is R-4-trimethylammonium-3-[tetradecylcarbamoyl)-aminobutyrate (ST1326).

18. A method of treating psoriasis in a mammalian subject comprising topically administering to the skin of the mammalian subject an effective amount of R-4-trimethylammonium-3-(tetradecylcarbamoyl)-aminobutyrate (ST1326) or a pharmaceutically acceptable salt or prodrug thereof in a pharmaceutically acceptable carrier.

19. The method of claim 15, wherein the mammalian subject is a human subject.

20. The method of any of claim 15, further comprising administering a steroid to the mammalian subject.
